(19) United States
(12) Patent Application Publication
(10) Pub. No.: US 2020/0076751 A1
(43) Pub. Date: Mar. 5, 2020

(54) A Method and System for Expressing Oneself

(71) Applicant: RAHUL YE. SHETYE, PEN (IN)

(72) Inventor: RAHUL YE. SHETYE, PEN (IN)

(21) Appl. No.: 16/489,453

(22) PCT Filed: Feb. 28, 2018
(86) PCT No.: PCT/IN2018/050105

§ 371 Date: Aug. 28, 2019

(30) Foreign Application Priority Data
Feb. 28, 2017 (IN) 201621029801

(51) Int. Cl. H04L 12/58; G16H 20/70

(52) U.S. Cl. CPC H04L 51/046; H04L 51/32; H04L 51/10; G16H 20/70

(57) ABSTRACT

The present invention relates to a simplified system and method thereof for expressing oneself, by creating interactive method and system platform providing a Plurality of users to express themselves through at least one single screen user interface that provides a user through at least one process to nurse & recover his/her broken heart and shattered self-confidence thus reviving themselves.

### BACKGROUND

### A. Technical Field

The present invention relates to a method and system for Expressing oneself, more particularly the invention relates to a creating interactive method and system platform providing a Plurality of users to express themselves through at least one single screen user interface that provides a user through at least one process an opportunity to revive themselves.

### B. Background of the Invention

World is full of Pain, Grief and Losers. Wiseman says Joy & Happiness is just a Myth. But still we always celebrate good moments, memories, joy, happiness and winners.

Over few decades or so, the effective user interaction technology has developed tremendously. This had resulted into technological implementation of various methods to support at least one user to be in happy and joyful company to help the at least one user to nurse & recover his/her broken heart and shattered self-confidence.

However, it is generally observed that people passing through low phase are cheered by others to revive them but instead of getting out of it they tend to go deeper in their pain and grief. They generally develop a feeling that their world is full of Pain, Grief, Sadness, Mental Traumas, Miseries, Misfortune & Tragedies and consider themselves world's the most unhappy, unlucky person and a complete Loser.

For the reasons stated above, which will become apparent to those skilled in the art upon reading and understanding the specification, there is a need in the art for a system and method for Expressing oneself that creates interactive method and system platform providing a Plurality of users to express themselves through at least one single screen user interface that provides a user through at least one process to revive themselves that is useable, scalable and independent of new technology platforms, uses minimum resources that is easy and cost effectively maintained and is portable and can be deployed anywhere in very little time.

### SUMMARY OF THE INVENTION

The present invention relates to a simplified system and method thereof for expressing oneself, by creating interactive method and system platform providing a Plurality of users to express themselves through at least one single screen user interface that provides a user through at least one process to nurse & recover his/her broken heart and shattered self-confidence thus reviving themselves.

Further the invention relates to a system and method thereof that give voice to all those miserable less fortunate and unlucky souls by creating Communication Bridge between them. By giving them dedicated platforms to discuss their plights and frustrations the system and the method there of help them get over melancholy.

### BRIEF DESCRIPTION OF THE DRAWINGS

FIG. 1 depicts PGL system technical landscape.

FIG. 2 describes the login process flow.

FIG. 3 describes the registration process flow.

FIG. 4 describes the user mourner navigation flow.

FIG. 5 describes the user mourner navigation flow.

### DETAILED DESCRIPTION OF THE PREFERRED EMBODIMENTS OF THE INVENTION

The invention described herein is directed to a simplified system and method thereof for expressing oneself, by creating interactive method and system platform providing a Plurality of users to express themselves through at least one single screen user interface that provides a user through at least one process to nurse & recover his/her broken heart and shattered self-confidence thus reviving themselves.

The embodiments herein provide a method and system for expressing one selves wherein a Plurality of users to express themselves through at least one single screen user interface that guides a plurality of user through at least one process by merging a plurality of user input controls with in the flow to provide examples, information, and motivation in timed sequence with the display of the plurality of user inputs that provides a user through at least one process an opportunity to revive themselves. Further the embodiments may be easily implemented in various data and information management structures. The method of the invention may also be implemented as application performed by a stand alone or embedded system.

The invention described herein is explained using specific exemplary details for better understanding. However, the invention disclosed can be worked on by a person skilled in the art without the use of these specific details.

References in the specification to “one embodiment” or “an embodiment” means that a particular feature, structure, characteristic, or function described in connection with the embodiment is included in at least one embodiment of the invention. The appearances of the phrase “in one embodiment” in various places in the specification are not necessarily all referring to the same embodiment.

Hereinafter, the preferred embodiments of the present invention will be described in detail. For clear description of the present invention, known constructions, and functions will be omitted.

Parts of the description may be presented in terms of operations performed by a computer system, using terms such as data, state, link, fault, packet, FTP and the like, consistent with the manner commonly employed by those skilled in the art to convey the substance of their work to others skilled in the art. As is well understood by those skilled in the art, these quantities take the form of data stored/transferred in the form of electrical, magnetic, or optical signals capable of being stored, transferred, combined, and otherwise manipulated through mechanical and electrical components of the computer system; and the term computer system includes general purpose as well as special purpose data processing machines, switches, and the like, that are standalone, adjunct or embedded.

According to an embodiment, the present invention, relates to dynamic interactive driven flows, and more particularly to a system and method thereof for expressing oneself a plurality of application controls in at least one single screen so that a plurality of users can interact with the system and method thereof to share, extract and process content that may include information, examples, encouragement and other means to maximize the likelihood that a user will successfully revive themselves through attending at least one process.

Creating a simple interface for users is a critical need for any system that intends to build wide adoption. This need is particularly true when users are exposed to the system and the process implemented thereof for the first time to complete specific steps in order to sign up, set up, explore and use the system. The need is amplified when the steps needed in initial stages are complex, and/or, the users might have a limited amount of time, attention, or background information needed to complete the steps. In such cases creating a user experience that helps the user to complete the process is essential.

An additional factor for new systems is that not only does the process need to be easy to adopt and execute, but in many cases the user needs some motivation to encourage them to take the time and effort to complete a sign up and setup process. This level of motivation is as much an emotional component to completion as is the technical simplicity.

Pain, Grief, Sadness, Mental Traumas, Miseries, Misfortune, Tragedies and Losers are terms people like to hide. These things are seldom discussed and always looked down upon. In its simplest embodiment, the invention give voice to all those miserable less fortunate and unlucky souls by creating Communication Bridge between them. By giving them dedicated platforms to discuss their plights and frustrations the system and the method thereof help them get over melancholy.

One of the embodiment of this invention would be a web page that includes one or more viewer input controls such as buttons or form fields, the interactive video player, a video file, and an instruction set.

Expanding upon one of the preferred embodiment, specialized instructions in the instruction set can allow the user to set user input component to coordinate to the at least one another user.

People share joy and success with others whom they know or do not know and feel proud of themselves on their achievement. Most lack in sharing their Pain, Grief and Losses as they feel their image shall be impacted. As per one of the embodiment of the present invention, the system and the method thereof may provide them the chance to share their pain, grief and losses and explore their unknown (unknown from others and at times from self, which one realize after introspection) image, hidden existence and explore the route to self-esteem and moral spirits by unfolding the hidden existence of self and meeting such like sufferers through anonymity.

As per one of the embodiment of the present invention, the system and the method thereof may provide a conversational platform. The conversational platform provided by various interface like Chat, Testimonial, Inner Pain Wall to disclose his/her Pain, Grief and Loser events to the unknown world.

As per one of the embodiment of the present invention, the system and the method thereof may allow a plurality of users to explore their unknown with other unknowns through anonymity.

As per one of the embodiment of the present invention, the system and the method thereof may allow a plurality of users to brighten their dark side by keeping themselves in dark in this dark unknown world of anonymity and explore the inebriating feel of darkness for self-enlightenment and for others in their TEAM DARK. Thus, allowing a plurality of user to attend a journey from darkness to enlightenment.

As per one of the embodiment of the present invention generally, the suffering people/user are of the opinion that their world is full of Pain, Grief, Sadness, Mental Traumas, Miseries, Misfortune & Tragedies. They generally consider themselves world's the most unhappy, unlucky person and a complete Loser until they came across several others who are just like them. After meeting, chatting and communicating with them they discovered that their Pain, Grief, Sadness, Mental Traumas, Miseries, Misfortune and Tragedies are nothing compare to the others.

As per one of the embodiment of the present invention, the system and the method thereof is specially designed for People suffering Pain, Grief, Sadness, Miseries, Traumas, Misfortune and Tragedies in their respective lives. By exploring the system people who consider themselves Depressed, Sad and Unlucky can chat directly with similar set of people around the world who are facing difficulties in their lives and are in deep state of Sorrow.

As per one of the embodiment of the present invention, the system and the method thereof may make the users to realize that others hardly know about themselves. For example, if a user may ask himself or herself how much others know about them they may get answer as
Their wife 25%
Their husband 25%
Their siblings 30%
Their parents 40%
Their friends 50%

Still a user in the example keep 50% to themselves and seldom speak or express this part.

They always carry this excess baggage wherever you go and find it difficult to share this part with anyone. This leads to stress loneliness and as a result make you erratic. By remaining anonymous this system and the method thereof gives the users chance to express their undisclosed 50%. They can share their undisclosed Pain, Grief, sadness, problems, secrets and complaints in appropriately chosen interactive interfaces provided by the system and the method thereof such as chat windows. This will definitely take away the burden on their mind and make them feel really good

As per one of the embodiment of the present invention, the system and method thereof may provide exposure to its user to log into dedicated chat windows such as but not limited to chat windows classified like below and help them chat with People facing and passing through similar circumstances around the world with options like Gender, Age, Country, State, City and Languages.
1. Pain
2. Grief
3. Sorrows
4. Sadness
5. Miseries
6. Mental Traumas
7. Physical Traumas
8. Misfortunes
9. Tragedies
10. Losers
11. Lost a Job
12. Jobless
13. Poverty
14. Debt ridden
15. Mental Sicknesses
16. Fears and Phobias
17. Unlucky
18. Heartbroken and love lost
19. Loss of the dear ones

### Sub Categories

A) Parents
B) Spouse
C) Children
D) Friend
20. Marital Discontents

### Sub Categories

A) Can't get along with your husband/wife/partner
B) Can't get along with your in-laws
C) Dowry
D) Violence
E) Extra marital
21. Parental Discontents
22. Peer pressure
23. Terminally ill patients
24. Old age
25. Handicapped
26. Divorced
27. Insecurities
28. Low self-esteem
29. Low on confidence
30. Depression
31. Frustration
32. Corruption
33. Discrimination
34. Helplessness
35. Violence
36. Addiction
37. Feeling deprived
38. Feeling lonely and loners
39. Accidents
40. Overweight & Underweight
41. Casteism
42. Racism,
etc. . . .

And many others specially created by chat participants as per their mental state and circumstances

As per preferred embodiment of the present invention the plurality of user's/participant's original identities will be hidden for obvious reasons and they all will be given specific user id by the system and the method thereof.

It is generally observed that people passing through low phase are cheered by others to revive them but instead of getting out of it they tend to go deeper in their pain and grief.

As per preferred embodiment of the present invention, Contrary to belief that happy and joyful company help you nurse & recover your broken heart and shattered self-confidence many times it actually end up hurting the person more. People going through Pain, Grief, Sadness, Mental Traumas, Miseries, Misfortune, and Tragedies get along better with people passing through similar circumstances. As all of them are passing through similar set of mindset and events, they find it easy to assess pain grief and sorrows of each other. It gives them a feeling they are not alone and not the only one going through these traumas and miseries. Thus, it built unseen bond between them and other counterparts. And it actually helps them face their adversity better and brings them to peace.

To support the above concept and make it a reality, the system and the method of the present invention make the conversation easy between mourners over their pain, grief and losses. The system of the present invention can be assessed seamlessly over internet across most gadgets like mobile phones, Tablets and Personal Computers.

As per one of the embodiment of the present invention the system and the method thereof may be based on the Client-Server Architecture. The Server component may be servers in a clustered form, thereby increasing the scalability to adapt to demand from time-to-time. The database may be used for the purpose of storing conversation among the mourners. It may implement the Clustered and Failover mechanism for high availability.

As per one of the preferred embodiment of the present invention, the system and the method thereof differs with current market available similar products for different purpose:
It is designed for the mourners. But people are free to share their success and reveal how they overcame their fears, pains, losses and grief, in the testimonial section. The ultimate objective of the present invention is to provide platform for mourners and learn from each other and build each others confidence and move ahead successful in life.
Unlike other social networking platforms and messengers such as but not limited to Whatsapp, the system and the method thereof allows the user/mourners hide their identity and seek and give strengths to others. Aiming to build up the trust and other abstract qualities to build a new social environment. This targets another belief—that unknowing the unknown through anonymity can lead to successful social life and rise above the rest.
Unlike other social networking platforms and messengers such as but not limited to Whatsapp, according to the system and the method thereof the user mourners shall get a unique id, by generating the unique id through registration process for transacting in this virtual world with real people flocked together with similar pain, grief and losses. Mourners are free to upload any image—that does not reveal their identity. They, can choose from the various Avatars from the gallery.
The PGL system interface (Light PGL)/app icon can be changed by user, in order to maintain secrecy. This feature shall free the user mourners from getting noticed accidentally among the physical world, in case, if anyone happen to see his/her phone app icons. Thus, the system and the method thereof help the user mourners to overcome this social stigma and setback and gain confidence to fight the “PAIN, GRIEF & Losses”—just like few eminent members of society.
Unlike other social networking platforms and messengers such as but not limited to Whatsapp, the technical difference in the system and the method thereof is that user do not have any past conversations saved offline and on user's device. This feature is security feature as the system and the method thereof value data privacy and shall try to secure the system in it's architecture as much as possible. The user can, upon special request have this Immediate history feature where they can view the records for past 2 months or 500 conversations whichever is less, through the Immediate history feature
The system and method thereof adopts the SMS confirmation of key for new sign-ups.
According to the system and method thereof for sign-up purpose, user can login using the third party services like login using the facebook or gmail. The system and the method thereof provides this feature keeping in mind that people do not have to remember too many userid/password combination.
The system and the method thereof provides add-on sign-up method wherein everytime user mourner sign-up, they get a 4 character public key and at the time of registration the 4 character private key is setup by user mourner. The combination helps user to login successfully.
The system and method thereof provide advantage as a light weight smart phone system, light to install and maintenance free from memory perspective. (No more deleting your conversations manually).
The system and method thereof is configured to implement service features viz analytics, cloud messaging, invites, dynamic links, remote configs, predictions, advertising on mobile and online advertising service.

As per one of the embodiment of the present invention the limitation of the system and the method thereof may be listed as:
Any Light PGL can communicate with another Light PGL or PGL App and vice versa.
Immediate history feature that allows mourner to view past 500 conversations or conversations of the past 2 months in each of his/her/their conversation
Mourner can flock other mourner. (It is similar to like on facebook, with a difference—that you mark another mourner to be of the same suffering category—there by increasing the conversation among the flocked mourners and take conversations to a new level.
Only mourners flocked in particular category can form a group. The mourner who started the group can impart admin rights to other mourners who subscribe. Any mourner can unsubscribe from group at any time.
Since all mourners are adult and the system respect netizens of PGL to describe the sanity social rules, any objectionable material—photo, content, testimonial can be reported as abuse by any mourner. Once any material is reported as abuse, that can not be viewed by other users. Such user who posted abuse, is immediately suspended for a months time. A warning is issued to such user. If 3 warnings are received by any user, such user will be permanently suspended from accessing the PGL system for lifetime.
Testimonial section where a mourner can mark his/her category of pain, grief, losses and write a short write-up—similar to Testimonial. We believe that this testimonial is the binding factor for mourners to connect and start conversation and share their experiences and feel light and explore path to success and relief.
Relief Wall: This feature helps mourner (rather no more mourner—but a guide now) to proclaim how he/she/they have learned from each other's experience and made a historical relief or success. On the relief wall one can acknowledge other mourners and those who are marked by guide (previously known as mourner) will be sent an invite for acknowledgement. Once you have guided more than five mourners to the path of success through your experience and acknowledged by these 5 mourners—you attain a Preacher role (A member advancement from a mourner Guide→Preacher)

As per one of the embodiment of the present invention the system may comprise of:
Registration unit: Takes a details from user such as Last Name, First name, Country, State, Pin(Zip Code), age, email Id, mobile number from user. In this module it shall also ask for a 4 digit private key to be entered by user. This 4 digit are the private key part of password for all successful login attempts. The remaining 4 digits/char/alphanumeric shall be sent to registered phone number as sms or as content in registered emailed. It generates a unique mourner Id—A unique system generated Id for the user (hereby identified as mourner). Mourners can select and upload an image that shall be displayed along with the mourner id.
Login unit: receives users unique mourners id through the login interface provided through light PGL at user device and sends it to the server and is then notified to other already logged in Mourners. Any offline messages from previously left conversations can be checked. Alternative to Registration from mobile is authentication through already used login id's of the user at other social networking platforms such as google authentication shall be enabled to Login directly.
Chat Groups unit: —Standard and Customized: Standard Chat Group is the one created or approved by the PGL Introspection Team after being nominated by 50%+customized group mourners of that group. Standard Chat Groups are initiated category by the PGL Introspection team. To which any member can join and move away at any time and can do it repeatedly.
Customized Chats shall get a checkbox enabled for nomination for promoting it to the Standard Chat Group. Once 50%+mourners of this group tick the checkbox the group shall get notification that their request is under accepted for processing by PGL Introspection Team.
Language independent chats—for multilingual chats for example chat in different languages such as but not limited to French, Spanish, English and German. A mourner can chat in French and communicate with another person who understands either of English, Spanish or German Language through the language converter unit implemented at the system.

As per one of the embodiment of the present invention the system and method thereof may have a special series of Emojis developed considering each category like fear, type of losses—action event based. eg: a feary face, a scared face, distress man sitting with heads down., etc.

As per one of the embodiment of the present invention the system and method thereof for security measures and to track any misuse of platform, in some interval the system and the method thereof seek the location details and ip address from the chat device. Just in case if the ip address jumping pattern is observed the alert will be sent to the PGL monitoring system for further appropriate measures

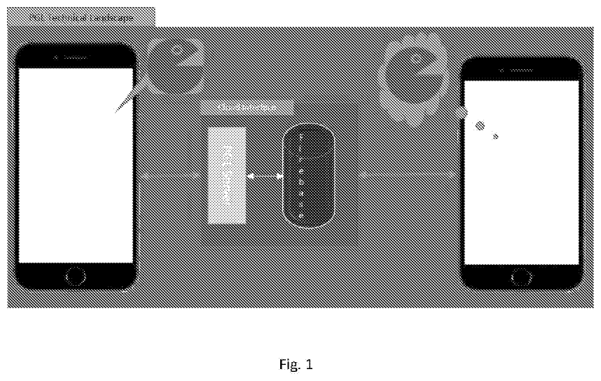

The FIG. 1 depicts PGL system technical landscape according to one of the embodiment of the present invention. The system broadly comprise of the plurality of user devices connected through at least one telecommunication network implementing at least one compatible network protocol on a cloud interface through the PGL firebase to the PGL server thus implementing the Client-Server Architecture. The Server component may be servers in a clustered form, thereby increasing the scalability to adapt to demand from time-to-time. The database may be used for the purpose of storing conversation among the mourners. It may implement the Clustered and Failover mechanism for high availability.

According to a preferred embodiment of the present invention a system for expressing oneself comprises a plurality of user Devices;
at least one server connected to the plurality of user devices; at least one network; at least one database to store all data related to users and the associated functionalities of the system; at least one cloud computing platform connecting the at least one server over the at least one network; wherein a processor of the user device is configured through a PGL system interface (Light PGL) unit to provide various interactive interfaces such as chat, testimonials, Inner pain wall, login interface, registration interface at the user device; wherein the at least one server comprises a registration unit, the registration unit comprise of a processor and a registration input extraction unit, is configured to perform the registration process of the user mourners; a login unit, the login unit comprise of a processor and a login input extraction unit, is configured to perform the login process of the user mourners; a chat group unit, the chat group unit comprise of a processor and a database, is configured to perform the process of chat group creation, maintenance and upgradation of the chat groups and storing of the chat at the database; a language converter unit, the language converter unit comprise of processor and linguistic database, is configured to convert the communication, data, chat etc. from one language to other preferred language of the user mourners; a PGL monitoring system unit comprise of a processor, an artificial intelligence unit consisting of artificial intelligence processor performing analytics and predictions, a processor configured to coordinate with the artificial intelligence unit and perform cloud messaging, invites, dynamic links, remote configs, advertising on mobile, online advertising service a database and other associated functionalities; and a central processing and control unit for coordinating the various units constituting the system.

According to a preferred embodiment of the present invention a method for expressing oneself comprising steps of Identifying at least one register user; authenticating and authorizing the at least one register user; obtaining the at least one register user details; directing the user to the registration interface at the light PGL in case of failure of authentication or new user; allowing the authenticated user to navigate through the PGL world; monitoring the actions of the user mourners to provide suggestions and/or validate the objection and provides detailed report and recommend action and preventive measures.

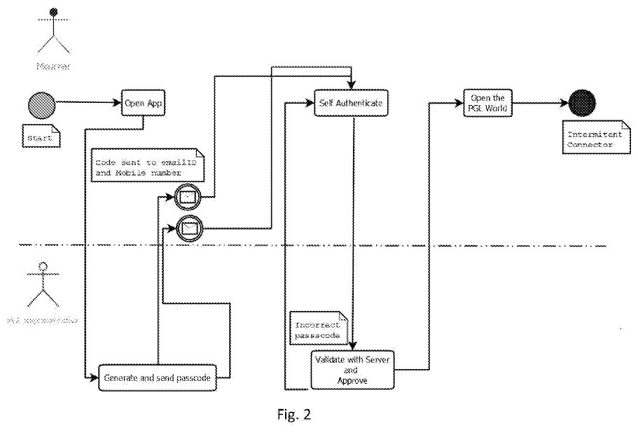

FIG. 2 describes the login process flow wherein the user mourners provides his/her unique id for transacting in this virtual world through the light PGL login interface with real people flocked together. Alternatively, user can login using the third party services like login using the facebook or gmail. Everytime user mourner sign-up, they get a 4 character public key and at the time of registration the 4 character private key is setup by user mourner. The combination helps user to login successfully. The system and method thereof adopts the SMS/email id confirmation of passcode/key for login/sign-ups. The user mourner is subjected to self-authentication by providing the passcode which is validated at the login unit at the server and further to the approval the PGL world is opened/access to the PGL world is provided to the user.

In case the user fails the validation, or the user is new user, the user is directed to the registration interface at the light PGL.

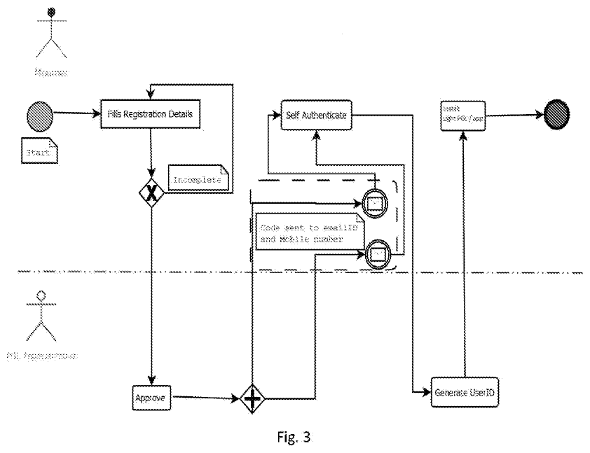

FIG. 3 describes the registration process flow wherein the user mourners provides his/her details such as Last Name, First name, Country, State, Pin (Zip Code), age, email Id, mobile number through the registration interface of the light PGL to the registration unit at the server. If the registration unit finds the registration details incomplete, it prompts the user to complete the details. In this module it shall also ask for a 4 digit private key to be entered by user. This 4 digit are the private key part of password for all successful login attempts. Upon approval of the details a passcode is generated. The system and method thereof adopts the SMS/email id confirmation of key for login /sign-ups. The user mourner is subjected to self-authentication by providing the passcode which is validated at the registration unit at the server and further to the approval a unique User ID is generated by the registration unit. Further to the generation of the unique User ID user is allowed to install the PGL system interface (light PGL)/app on the user device.

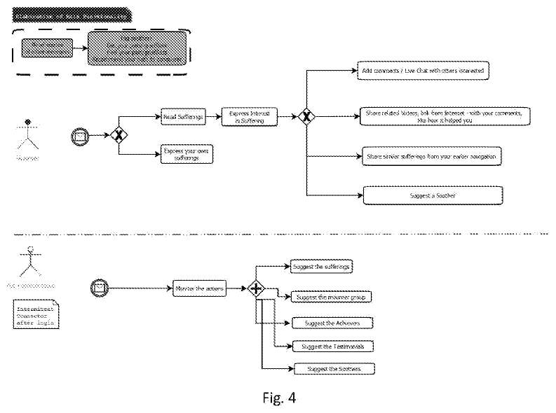

FIG. 4 describes the user mourner navigation flow wherein further to login the user mourners can select the appropriate category of the suffering and express his/her own sufferings. Alternatively, the user mourners can read the sufferings posted by other mourners, express his/her interest in the sufferings may be by adding comments or through live chat with others interested, may share related videos, links from internet with his/her comments like how it helped her/him, may share similar sufferings from her/his earlier navigation or suggest a soother. The PGL monitoring system unit monitors the actions of user mourners and may suggest the appropriate category of the sufferings, may suggest the appropriate mourner group(s), suggest the achievers, suggest the testimonials, suggest the soothers etc.

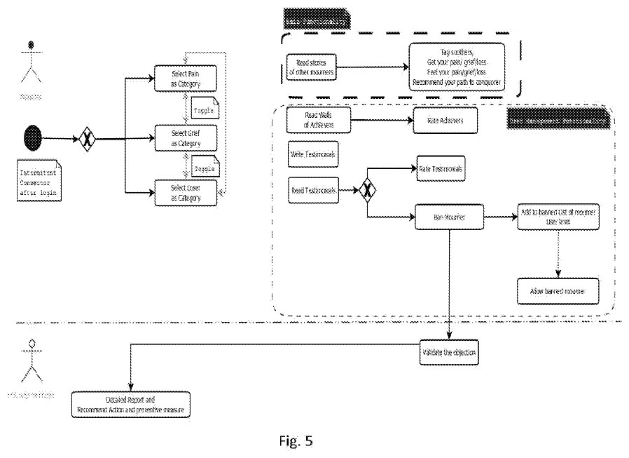

FIG. 5 describes the user mourner navigation flow wherein further to login the user mourners can select Pain or Grief or looser as category or can toggle among these and their subcategories. Further, the user mourner can read stories of other mourners, can tag the soothers, get her/his pain /grief/loss recognized, feel her/his pain /grief/loss, recommend her/his path to conquer. Further, the user mourner can read walls of the achievers, rate the achievers, write testimonials, read testimonials and depending on the testimonial may rate the testimonial or ban mourner and add to banned list of mourner User level or alternatively allow the banned mourner. The PGL monitoring system unit monitors the actions of user mourners and in case a User mourner bans any mourner, the PGL monitoring system validate the objection and provides detailed report and recommend action and preventive measures.

As per one of the preferred embodiment of the present invention, the system and the method thereof wherein at least one set of rules/instructions, which when applied, causes the processor to provide with a perspective based on the given data which will be instrumental in providing dynamic interactive single screen user interface. The system may be able to refresh all the content previously generated using freshly available data on a periodic basis.

In some examples, the systems described herein, may include one or more processors, one or more forms of memory, one or more input devices/interfaces, one or more output devices/interfaces, and machine-readable instructions that when executed by the one or more processors cause the system to carry out the various operations, tasks, capabilities, etc., described above.

In some embodiments, the disclosed techniques can be implemented, at least in part, by computer program instructions encoded on a non-transitory computer-readable storage media in a machine-readable format, or on other non-transitory media or articles of manufacture. Such computing systems (and non-transitory computer-readable program instructions) can be configured according to at least some embodiments presented herein, including the processes described herein.

The programming instructions can be, for example, computer executable and/or logic implemented instructions. In some examples, a computing device is configured to provide various operations, functions, or actions in response to the programming instructions conveyed to the computing device by one or more of the computer readable medium, the computer recordable medium, and/or the communications medium. The non-transitory computer readable medium can also be distributed among multiple data storage elements, which could be remotely located from each other. The computing device that executes some or all of the stored instructions can be a microfabrication controller, or another computing platform. Alternatively, the computing device that executes some or all of the stored instructions could be remotely located computer system, such as a server.

Further, while one or more operations have been described as being performed by or otherwise related to certain modules, devices, or entities, the operations may be performed by or otherwise related to any module, device, or entity. As such, any function or operation that has been described as being performed by the device could alternatively be performed by a different server, by the cloud computing platform, or a combination thereof.

Further, the operations need not be performed in the disclosed order, although in some examples, an order may be preferred. Also, not all functions need to be performed to achieve the desired advantages of the disclosed system and method, and therefore not all functions are required.

While select examples of the disclosed system and method have been described, alterations and permutations of these examples will be apparent to those of ordinary skill in the art. Other changes, substitutions, and alterations are also possible without departing from the disclosed system and method in its broader aspects as set forth in the above description.

In view of the many different embodiments to which the above-described inventive concepts may be applied, it should be recognized that the detailed embodiments are illustrative only and should not be taken as limiting the scope of our invention. Rather, we claim as our invention all such modifications as come within the scope and spirit of the above description.

The present invention may overcome the challenges of the current scenario through the described system and method for creating interactive user workflows through a single screen user interface that guides a user through a software workflow process by merging user input controls with in the flow of a video and uses the video content to provide examples, information, and motivation in timed sequence with the display of user input controls.

1.-33. (canceled)

34. A system for expressing oneself, comprising:
a user device;
a server connected to the user device;
a network;
a database to store data related to users;
a cloud computing platform connecting the server over the network;
wherein a processor of the user device is configured through a PGL system interface unit to provide interactive interfaces including any of chat, testimonials, inner pain wall, login interface, and registration interface at the user device;
wherein the server comprises:
a registration unit comprising a processor and a registration input extraction unit, and is configured to perform a registration process of user mourners;
a login unit comprising a processor and a login input extraction unit, and is configured to perform a login process of the user mourners;
a chat group unit comprising a processor and a database, and is configured to perform a process of chat group creation, maintenance and upgradation of the chat groups, and storing of chats at the database;
a language converter unit comprising a processor and linguistic database, and configured to convert communications, data or chats from one language to another language requested by the user mourners;
a PGL monitoring system unit comprising a processor, an artificial intelligence unit comprising an artificial intelligence processor performing analytics and predictions, and a processor configured to coordinate with the artificial intelligence unit and perform cloud messaging, invitations, dynamic links, remote configurations, advertising on mobile devices, online advertising, and servicing databases; and
a central processing and control unit for coordinating the units constituting the system.

35. The invention according to claim 34, wherein the registration unit takes details from the user mourner including any one of last name, first name, country, state, PIN (zip code), age, email ID, and mobile number from the user mourner, and asks for a four digit private key to be entered by the user mourner, wherein the four digits are the private key part of a password for all successful login attempts, and sends the remaining four digits/characters/alphanumeric to a registered phone number as an SMS or as content in a registered email and generates a unique mourner ID.

36. The invention according to claim 34, wherein the user mourners select and upload an image that is displayed along with the mourner ID.

37. The invention according to claim 34, wherein the login unit receives the unique user mourner ID through the login interface provided through a light PGL at a user mourner device and sends it to the server and is then notified to other already logged in user mourners.

38. The invention according to claim 34, wherein the chat group unit includes a standard chat group that is created or approved by a PGL introspection team after being nominated by 50% or more of customized group user mourners of that group to which any member can join and move away at any time and can do it repeatedly.

39. The invention according to claim 34, wherein the language converter unit facilitates multilingual chats in different languages including French, Spanish, English or German.

40. A method for expressing oneself, comprising the steps of:
identifying a registered user mourner;
authenticating and authorizing the registered user mourner;
obtaining the registered user mourner's details;
directing the user mourner to the registration interface at a light PGL in case of failure of authentication or a new user;
allowing the authenticated user mourner to navigate through a PGL world; and
monitoring the actions of user mourners to provide suggestions or validate an objection and providing reports and recommending action and preventive measures.

41. The invention according to claim 40, wherein the user mourner provides a unique ID for transacting in a virtual world through the light PGL login interface with real people flocked together.

42. The invention according to claim 40, wherein the user mourner can login using third party services including FACEBOOK or GMAIL.

43. The invention according to claim 40, wherein every time a user mourner signs up, they get a four character public key and, at the time of registration, the four character private key is setup by the user mourner and the combination aids the user mourner to login successfully.

44. The invention according to claim 40, further comprising the step of adopting an SMS or email ID confirmation of a passcode or key for login and sign ups.

45. The invention according to claim 40, wherein the user mourner is subjected to self-authentication by providing the passcode that is validated at the login unit at the server and further to approval of the PGL world is opened and access to the PGL world is provided to the user mourner.

46. The invention according to claim 40, wherein the user mourner provides details including any one of last name, first name, country, state, PIN (Zip Code), age, email ID, mobile number through the registration interface of the light PGL to the registration unit at the server.

47. The invention according to claim 40, wherein a module asks for a four digit private key to be entered by the user mourner, and the four digits are the private key part of a password for all successful login attempts.

48. The invention according to claim 40, wherein upon approval of the details, a passcode is generated, and further comprising the step of adopting an SMS or email ID confirmation of a passcode or key for login and sign ups.

49. The invention according to claim 40, wherein the user mourner is subjected to self-authentication by providing the passcode that is validated at the registration unit at the server and further to the approval a unique user ID is generated by the registration unit.

50. The invention according to claim 40, wherein the user mourner is allowed to install a PGL system interface on the user device.

51. The invention according to claim 40, wherein the user mourner can select an appropriate category of suffering and express their own sufferings.

52. The invention according to claim 40, wherein the user mourner can read the sufferings posted by other user mourners, express their interest in the sufferings by adding comments or through live chat with others, share related videos, links from the Internet with their comments, share similar sufferings from their earlier navigation, or suggest a soother.

53. The invention according to claim 40, wherein the PGL monitoring system unit monitors the actions of user mourners and suggests the appropriate category of the sufferings, suggest the appropriate mourner groups, suggest the achievers, suggest the testimonials, or suggest a soother.

54. The invention according to claim 40, wherein the user mourner can select pain or grief as a category or can toggle among these and their subcategories.

55. The invention according to claim 40, wherein the user mourner can read stories of other user mourners, can tag the soothers, get their pain, grief or loss recognized, feel their pain, grief or loss, or recommend their path to conquer.

56. The invention according to claim 40, wherein the user mourner can read walls of achievers, rate the achievers, write testimonials, read testimonials and, depending on the testimonial, rate the testimonial or ban a user mourner and add to a banned list of user mourners or, alternatively, allow the banned user mourner.

57. The invention according to claim 40, wherein the PGL monitoring system unit monitors the actions of user mourners and in case a user mourner bans any other user mourner, the PGL monitoring system validates the ban and provides a report and recommends action and preventive measures.

58. The invention according to claim 40, wherein any light PGL can communicate with another light PGL or PGL application and vice versa.

59. The invention according to claim 40, wherein an immediate history feature allows a user mourner to view the past five hundred conversations or, alternatively, conversations of the past two months in each of their conversations.

60. The invention according to claim 40, wherein a user mourner can flock to other user mourners, wherein the user mourner marks another user mourner to be of the same suffering category, thereby increasing conversation among the flocked user mourners.

61. The invention according to claim 40, wherein only user mourners flocked in a particular category can form a group and the user mourner that started the group can impart administrative rights to other user mourners that subscribe, wherein any user mourner can unsubscribe from the group at any time.

62. The invention according to claim 40, wherein once any material is reported as abuse, that material cannot be viewed by other user mourners and such user mourner that posted abuse, is immediately suspended.

63. The invention according to claim 40, wherein a testimonial section is provided, wherein a user mourner can mark their category of pain, grief, or loss and post a testimonial.

64. The invention according to claim 40, wherein a relief wall is provided, wherein the relief wall helps users mourners to express how they have learned from each other user mourner experiences and make a historical relief or success, wherein on the relief wall one can acknowledge other user mourners, and those that are marked will be sent an invitation for acknowledgement.

65. The invention according to claim 40, further comprising a series of emoji's developed for categories including fear, type of loss, or action events.

66. The invention according to claim 40, wherein the user mourners can hide their identity and seek and give strength to others.
